(19) United States
(12) Patent Application Publication
(10) Pub. No.: US 2009/0131267 A1
(43) Pub. Date: May 21, 2009

(54) USE OF POLYMERS FOR INCREASING THE SIGNAL INTENSITY WHEN CARRYING OUT DETECTION REACTIONS

(76) Inventors: Peter Porschewski, Langenfeld (DE); Kerstin Steinert, Langenfeld (DE)

Correspondence Address: Baker Donelson Bearman, Caldwell & Berkowitz, PC, 555 Eleventh Street, NW, Sixth Floor, Washington, DC, 20004, US

(21) Appl. No.: 12/159,977

(22) PCT Filed: Jan. 3, 2007
(86) PCT No.: PCT/EP07/50033

§ 371 Date: Dec. 29, 2008

(30) Foreign Application Priority Data
Jan. 3, 2006 (DE) 10 2006 000 707.7

(51) Int. Cl. C40B 30/04; G01N 33/545

(52) U.S. Cl. 506/9; 436/531

(57) ABSTRACT

The present invention relates to increasing the signal intensity while simultaneously reducing unspecific background binding when carrying out detection reactions, in particular immunoassays by using polyvinyl derivatives.

The present invention relates to increasing the signal intensity while simultaneously reducing unspecific background binding when carrying out detection reactions, in particular immunoassays.

Immunoassays are widely used inter alia in analysis, medical diagnostics and human therapy. For example, stored blood is routinely examined for relevant pathogens with the aid of ELISAs and antibodies in medical analytical laboratories. Polyclonal antibodies—a mixture of antibodies with different binding epitopes—are most frequently used in this connection. In recent years, repeated attempts have been observed to establish in addition to the proven ELISA relatively new methods in diagnostics which are said to be distinguished by higher sensitivity, lower costs and shorter processing times. The prior art has thus disclosed developments of developing detection techniques based on the “Luminex-xMap” technology which as far as possible meet the requirements illustrated above.

The Luminex-xMAP technology is based on spherical polymeric particles on the microscopic scale, in particular polystyrene particles, usually referred to as microspheres or beads in the prior art. Inside said particles there are at least two different fluorescent dyes. The variation of different proportions of the two dyes here defines populations of beads which can be spectrally distinguished unambiguously. As a result, for example, one hundred different microsphere types are available. Each species of the various beads can be coated with a specific detection reagent or antigen by means of simple coupling chemistry. In this way it is theoretically possible to carry out up to one hundred different detection reactions in a sample simultaneously. By 2004, approx. 30 of these detection reactions had already been established.

During said incubation with a few microliters of a sample, each microsphere population with the appropriately coated detection reagent binds to its specific target molecule. The target molecules bound to the surface of the microspheres are recognized by a specific detection molecule which—in the example illustrated—carries a green fluorescent marker, also referred to as reporter.

Thus it is possible, for example, to individually evaluate thousands of microspheres in a Luminex 100 analyzer within seconds. A first laser in the instrument is used for exciting the red fluorescent dyes inside the beads, which are classified on the basis of their fluorescence emission. A second laser—in the present example—excites, for example, the green fluorescent dye of the reporter, and the intensity of the emitted light is recorded. Thus the identity of the antigen is specified with the aid of the first laser, while the second laser enables the antibodies specifically bound to the particular antigen to be detected qualitatively and quantitatively.

Using this technology it is possible to carry out up to 9 600 diagnostic assays in a single 96-well microtiter plate inside 3 hours. The Luminex-xMAP technology therefore provides decisive advantages over conventional methods such as, for example, the known ELISA:
for example, detection in a well is carried out without washing steps, resulting in shorter processing times and lower costs;
only very small amounts of sample are required due to the very high sensitivity;
the process is also fully automatable.

Accordingly, the prior art has disclosed special immunoassays developed for the Luminex technology. These include in particular the LiquiChip immunoassays developed by Qiagen in Hilden, Germany.

The principle of a LiquiChip immunoassay is based on the use of an (ELISA) antibody sandwich pair on LiquiChip Beads. For this purpose, one antibody of the sandwich pair is covalently immobilized as capture molecule (capture antibody) to the surface of the polystyrene beads. The second antibody is used as antigen-specific primary antibody. This primary antibody may, for example, be biotinylated and may be detected with the aid of a streptavidin-phycoerythrin conjugate or by an appropriately (fluorophore) marker-conjugated species-specific detection antibody.

As in a Western blot or an ELISA, however, it is also possible in a LiquiChip immunoassay for individual sample or assay components to bind unspecifically to the matrix surface or to the immobilized capture antibody (background binding). Another problem relates to affinity, one of the physicochemical properties of the antibodies, for the particular biomarker. The affinity of the particular antibody codetermines the extent of binding of the biomarker in the sample, as has been disclosed in the prior art.

The range within which the biomarker is detectable and quantifiable is referred to here as dynamic range. Binding becomes saturated with increasing biomarker concentration. However, measurement at high concentrations beyond saturation is no longer possible. In contrast, low concentrations may likewise not be detected due to the low sensitivity or due to the unspecific background signal.

In order to quantify an analyte or biomarker, it is therefore important to cover as large a dynamic range as possible. This is especially important if only a small amount of starting material or sample is available.

It is therefore the object of the present invention to generate in the corresponding assays conditions which enable the bound analyte to produce a maximum signal—over a very large dynamic range—and which secondly allow only a minimum background signal to be produced at the same time.

In summary, the present invention therefore has the object to make possible an improved signal-background (signal/background, S/B) ratio.

Although the prior art has disclosed the reduction of unspecific background binding by using “blocking agents” such as, for example, BSA (bovine serum albumin), gelatin, casein and/or in combination with various detergents (NP-40, Tween20, Triton X-100), the prior art has, on the other hand, also reported that a signal increase can be achieved in immunoassays essentially only by means of enzyme-mediated signal amplification methods. Proposed solutions of this kind include, for example, classical ELISA applications, ECL-based approaches (ElectroChemiLuminescence), RCAT (Rolling Circle Amplification Technology), and other methods which may also be applied to protein arrays.

Signal amplification may thus improve assay sensitivity, thereby resulting in a larger dynamic range and an improved signal/background ratio (S/B).

Another advantage is the possibility of carrying out assays with reduced sample amounts, due to achieving higher sensitivities, especially if only a small amount of sample starting material is available.

With regard to the solution to the problem illustrated above, the prior art has disclosed the possibility of employing proteinogenic substances, such as for example BSA, casein or gelatin, or non-proteinogenic substances, in particular detergents, to prevent antibodies or antigens from binding unspecifically to the matrix surface or to the capture antibody in Western blot or ELISA applications. Said substances are used in the corresponding reaction buffers.

However, the prior art has in addition also disclosed that the detection antibodies are capable of binding unspecifically to the capture antibodies.

The buffers used in the prior art are normally standard buffers such as PBS or Tris buffers (pH 7-7.8). However, detergents which are used in the corresponding buffers in order to reduce the unspecific bindings produced by protein-protein or protein-matrix interactions have the serious disadvantage of dissolving the specific antigen-antibody interactions or even being capable of denaturing antigen and antibody and thereby having ultimately an inactivating effect, if the concentration of said detergents is too high.

In view of the above-described prior art, the object on which the present invention is based is thus that of overcoming the disadvantages of the prior art illustrated and enabling immunoassays—LiquiChip immunoassays—to be optimized with respect to increase in signal intensity—with non-enzymatically mediated signal amplification—and with simultaneous reduction of unspecific background binding.

It is another object of the present invention to enable the signal intensity to be increased with simultaneous reduction of unspecific bindings on “planar protein arrays”, wherein bound analytes are detected on an essentially planar array.

Another object of the present invention is to produce assay conditions in a detection method, which in addition also enable a very high specific signal to be generated using small amounts of sample.

These objects are achieved by using polyvinyl derivatives which are soluble under the assay/method conditions. Polyvinyl derivatives mean in accordance with the invention any derivatives of a polymer or oligomer that have been obtained by polymerization of a monomer with a vinyl structure, including in particular polyvinyl alcohol (PVA) or polyvinylpyrrolidone (PVP). In addition, however, polyvinyl derivatives mean according to the invention also oligomers or polymers with ester partial structures, such as, for example, polyvinyl acetate, where appropriate partially saponified, and copolymers such as, for example, ethylene-vinyl acetate copolymers, (1-vinyl-2-pyrrolidone)-vinyl acetate copolymers, vinyl acetate-vinyl laurate copolymers.

Surprisingly, the presence of said polyvinyl derivatives, in particular polyvinyl alcohol (PVA) or polyvinylpyrrolidone (PVP), in the reaction buffers was found to support the formation of specific antibody-antigen interactions—resulting in a higher signal—and apparently to be responsible for reducing unspecific interactions.

According to the invention, this results in an improved signal/background ratio over a larger dynamic range.

The use of soluble polyvinyl derivatives can therefore overcome the disadvantages of the prior art. In addition, soluble polyvinyl derivatives may be employed over a wider concentration range, compared to detergents, and effect an increase in signal intensity and sensitivity and at the same time even contribute to reducing unspecific bindings. —This results in an improved signal/background ratio over a larger dynamic range.

In principle, the use according to the invention of soluble polyvinyl derivatives such as, for example, polyvinyl alcohol (PVA) or polyvinylpyrrolidone (PVP) in the reaction buffers increases the signal intensities, while simultaneously reducing unspecific bindings, in all the immunoassays or ELISAs performed on hydrophobic surfaces.

Furthermore, it was surprisingly found that the use of polyvinyl derivatives may also improve the results of detection reactions carried out with a higher order of reaction (multiplex assay). According to the invention, the use of said polyvinyl derivatives opens up the possibility of enabling, due to the increase in sensitivity, even those parameters to be analyzed in parallel in this setup, whose signal intensities have been found to be too low under the previous conditions.

This is particularly interesting if it is intended to observe low expression analytes alongside high expression analytes simultaneously in one assay setup. The use according to the invention of said polyvinyl derivatives thus embodies another advantage over methods based on ELISA or Western blot which usually contemplate only one analyte.

Using these substances in addition not only results in a surprising improvement of assay results in immunoassays or ELISAs but also enables solubilized membrane proteins to be detected in an improved manner. Moreover, buffers having said polyvinyl derivatives improve in the abovementioned manner protein-protein and protein-DNA interaction assays and also DNA applications in LiquiChip assays on polystyrene surfaces.

Furthermore, said polyvinyl derivatives, in particular polyvinyl alcohol (PVA) and polyvinylpyrrolidone PVP, were surprisingly found to contribute to preventing undesired aggregation of the beads.

The present invention additionally enables the signal intensity to be increased—likewise with simultaneous reduction of unspecific bindings, when detecting bound analytes by means of “planar protein arrays”. In these protein arrays, the capture molecules are present on a support substance such as, for example, glass or glass slides, nitrocellulose, PVDF for polystyrene surfaces, embodying, for example, an antibody, a fragment of an antibody, an aptamer, a peptide or an anticalin.

However, using the abovementioned polyvinyl derivatives in the “LiquiChip immunoassays” has been found to be particularly advantageous. The principle of a LiquiChip immunoassay comprises the use of an (ELISA) antibody sandwich pair on LiquiChip beads. For this purpose, one antibody of said sandwich pair is covalently immobilized as capture molecule (capture antibody) to the surface of the polystyrene beads. Said beads are incubated with a sample (e.g. serum, lysate of cell cultures or tissue, cell culture supernatant) containing the corresponding antigen. After incubation the beads may optionally be washed. The specific antigen should remain bound to the beads via the capture antibody.

In a further step, the specifically bound antigen may be labeled, for example, with a second antigen-specific antibody and then be detected, for example using a fluorophore-labeled antibody directed against said second antibody. Each of these steps may result in other assay components (e.g. other proteins of the sample) binding unspecifically to the bead surface or to the capture antibodies.

To increase the signal intensity with simultaneous reduction of unspecific bindings, the capture-antibody beads are incubated with the sample (antigen) in a buffer which contains a soluble polyvinyl derivative.

Polyvinyl derivatives suitable for the solution of the object of the invention are any polyvinyl derivatives which are soluble or water-soluble under the defined reaction conditions. Preference is given to polyvinyl derivatives being, like for example polyvinyl-pyrrolidone and polyvinyl alcohol, in the form of their pure polymers, as copolymers with one another and/or with other suitable comonomers, and as mixtures.

Polymers and copolymers of vinyl alcohol or of its esters and of vinylpyrrolidone are well known to the skilled worker from the prior art, because they have been used for decades in numerous fields of daily life applications—for example as thickeners in the food industry and as excipients in the pharmaceutical and textile industries—to name but a few fields of use. With regard to the use of suitable copolymers, the use of any copolymer of vinyl alcohol or vinyl acetate and of vinylpyrrolidone as well as of copolymers of both—poly[vinylpyrrolidone-covinyl acetate] or poly[vinyl-pyrrolidone-covinyl alcohol] in addition to the copolymers already mentioned above—is possible, as long as they have a suitable solubility behavior in an, essentially, aqueous environment.

The particular molecular weights can be varied within wide limits for successful implementation of the method of the invention. Practically they are within a range from 5000 to 500 000 Da, preferably within a range from 24 000 to 360 000 Da, and particularly preferably from 31 000 to 186 000 Da. These ranges apply both to the individual polymers and to mixtures of two, three or more polymers, with mixtures of polyvinyl alcohol and polyvinylpyrrolidone being preferred.

A molecular weight range for the use of polyvinyl alcohol alone or in mixtures with polyvinylpyrrolidone is practically from 9000 to 500 000 Da, preferably 31 000 to 186 000 Da, and particularly preferably between 86 000 and 126 000 Da and between 146 000 and 186 000 Da.

A molecular weight range suitable for the use of polyvinylpyrrolidone alone or in combination with polyvinyl alcohol is practically from 5000 to 500 000 Da, preferably 10 000 to 450 000 Da and particularly preferably 24 000 to 360 000 Da.

The, individual or combined, concentration of the polyvinyl derivatives, for example in the LiquiChip immunoassays, is preferably within a range from 0.025 to 5% (w/v), preferably within a range from 0.025 to 2% (w/v) and particularly preferably within a range from 0.1 to 1% (w/v).

The beads are preincubated with the buffer containing the polyvinyl derivative, followed by the addition of the sample and a further incubation. However, the soluble polyvinyl derivatives, such as for example preferably polyvinyl alcohol or polyvinylpyrrolidone, may also be present in the particular buffers in the subsequent steps, such as for example during addition and incubation of the second antigen-specific antibody.

The present invention furthermore relates to a method of increasing the signal intensity with simultaneous reduction of unspecific bindings, which is characterized in that analytes bound to beads or to planar protein arrays are detected in the presence of a polyvinyl derivative. More specifically, the present invention relates to a method in which the capture molecules are applied to glass (glass slides), nitrocellulose, PVDF (polyvinylidene fluoride) or to polystyrene surfaces, and the detection reaction is carried out in the presence of a polyvinyl derivative.

The capture molecule here may be, for example, an antibody, the fragment of an antibody, an aptamer, a peptide or an anticalin.

In another embodiment, the present invention relates to a kit for carrying out an immunoassay, comprising at least one buffer with a polyvinyl derivative component. More specifically, the present invention in addition also relates to a kit, wherein the capture molecules are bound to the beads or to planar arrays. —Said beads or planar arrays may have a support material made of glass, nitrocellulose, PVDF (polyvinylidene fluoride) or polystyrene.

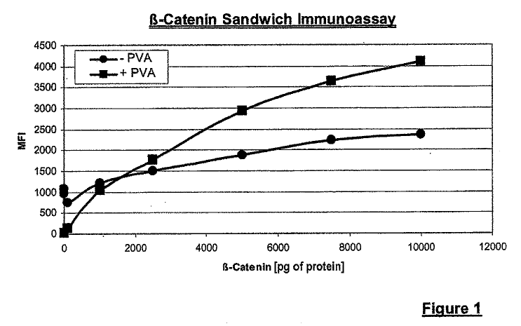

The positive effect of the use according to the invention of the polyvinyl derivatives can be proven experimentally in an impressive manner. —Thus, for example, the signal was markedly increased, while simultaneously unspecific background binding was reduced, in a monoplex sandwich immunoassay for detecting β-catenin (cf. protocol in Example 1). The graphical representation (FIG. 1) unambiguously shows that the signals stand out against the background, particularly in the lower part of the curve, with the use of the polyvinyl derivatives. Another feature that can be observed is that of signal values being obtained with the use of said polyvinyl derivatives even at lower protein concentrations, which values are reached in the curve without polyvinyl derivatives only at substantially higher protein concentrations. Thus, a value of approx. 2000 MFI (median fluorescence intensity) is obtained at a concentration of approx. 3000 pg of protein in the sample treated with polyvinyl alcohol, whereas this value can be achieved in the sample without polyvinyl alcohol only at a concentration of approx. 6000 pg of protein. In addition, it is apparent that a better signal/background (S/B) ratio can be achieved for all measured data using the method of the invention.

The examples below are intended to illustrate the invention and to support the findings illustrated above. Further advantageous embodiments are apparent from the examples.

### EXAMPLES

The following buffer solutions are used in the examples:

### Buffer Solutions:

Assay buffer I (with PVA or PVP):

| | |
|---|---|
| 10 mM NaH₂PO₄•H₂O | 1.4 g of NaH₂PO₄•H₂O |
| | (MW 137.99 g/mol) adjusted to |
| | pH 7.4 with sodium hydroxide |
| | solution |
| 150 mM NaCl | 8.77 g of NaCl (MW 58.44 g/mol) |
| 0.1% (w/v) BSA | 1 g of BSA |
| 0.02% (v/v) Tween ® 20 | 200 μl of Tween 20 |
| 2% (w/v) PVA | 20 g of polyvinyl alcohol |
| | (MW 86 000-126 000, 98-99% |
| | hydrolyzed) |
| or | |
| 2% (w/v) PVP | 20 g of polyvinylpyrrolidone |
| | (MW 40 000) |

Assay buffer II (does not contain PVA or PVP):

| | |
|---|---|
| 10 mM NaH₂PO₄ | 1.4 g of NaH₂PO₄•H₂O |
| | (MW 137.99 g/mol) adjusted to |
| | pH 7.4 with sodium hydroxide |
| | solution |
| 150 mM NaCl | 8.77 g of NaCl (MW 58.44 g/mol) |
| 0.1% (w/v) BSA | 1 g of BSA |
| 0.02% (v/v) Tween ® 20 | 200 μl of Tween 20 |

Sodium chloride, NaH₂PO₄.H₂O and the polyvinyl derivative were autoclaved in 900 ml of distilled water and dissolved in the process. The pH is then adjusted to 7.4 with sodium hydroxide solution (NaOH). This is followed by adding BSA and Tween 20 and bringing the total volume to 1 liter. The solution is filtered through a 0.45 μm filter prior to its use.

### Example 1

The experiments were carried out according to the following protocol:

In each well of a 96-well plate, the solutions below were pipetted into a filter plate. The filter plate is equilibrated with 50 μl of assay buffer I and the buffer is then removed with suction.

Subsequently, 30 μl of assay buffer I are added and admixed with 10 μl of bead solution (1250 beads, assay buffer II). This is followed by adding the sample in 10 μl of sample assay buffer II. The multiwell plate is then closed and incubated on a shaker (MTP Shaker) at 450 revolutions/min in the absence of light at room temperature (approx. 18-25° C.) over a period of 2 h (the incubation may optionally be carried out at 4° C. o/n). The buffer in the filter plate is then removed with suction and the beads are washed with 150 μl of assay buffer II. The latter is then again removed with suction and the beads are resuspended in 100 μl of assay buffer II. This is followed by adding 15-20 ng (up to 200 ng) of the second antigen-specific antibody in a volume of 10 μl of assay buffer II, and incubation at room temperature on a shaker at 450 revolutions/min over a period of 90 min. The addition of 200 ng of fluorophore-labeled detection antibody (optionally 100 ng of Streptavidin R Phycoerythrin [SAPE], when using a biotinylated antibody), in each case in a volume of 10 μl of assay buffer II, is followed again by incubation at room temperature on an MTP Shaker at 450 revolutions/min over a period of 30 min and subsequent analysis in a reader.

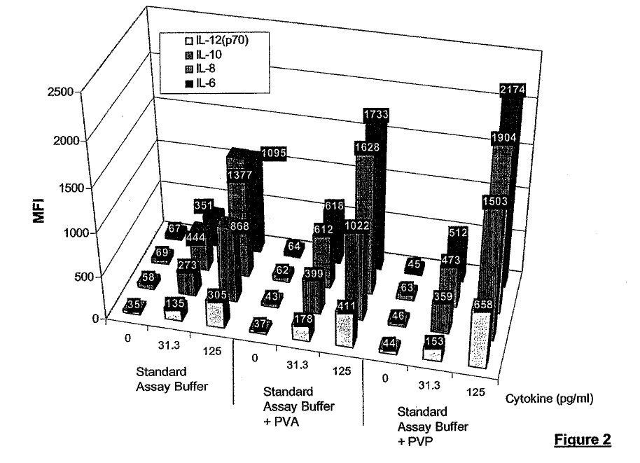

The influence of the polyvinyl derivatives on a multiplex immunoassay is depicted in FIG. 2 which shows the result of a cytokine multiplex sandwich immunoassay for detecting the interleukins IL-12, IL-10, IL 8 and IL-6, in which assay 1% (w/v) PVA (MW 86 000-126 000) or 1% (w/v) PVP (MW 40 000) in the buffer was used during the capture antibody/antigen reaction [the value bars for IL 12, IL 10, IL 8 and IL 6 in the three-dimensional diagram of FIG. 2 are arranged from the front to the back in the direction of viewing!]. This experiment also demonstrated that, alongside PVA, PVP also has a positive effect on background reduction and on increasing the signal intensity of the individual interleukins. In this experiment, the incubation buffer likewise contained the polymeric substances in the first step during binding of the antigens to the capture antibody beads.

### Example 2

The experiments were carried out according to the following protocol:

In each well of a 96-well plate, the solutions below were pipetted into a filter plate: The filter plate was first equilibrated with 50 μl of assay buffer I and the buffer was then removed with suction.

Then 50 μl of assay buffer I were first introduced and admixed with 25 μl of sample dissolved in assay buffer II. This is followed by adding 25 μl of assay buffer II containing in each case 2500 beads per cytokine (assay buffer II). The multiwell plate is then closed and incubated on a shaker (MTP Shaker) at 450 revolutions/min in the absence of light at room temperature (approx. 18-25° C.) over a period of 2 h (the incubation may optionally be carried out at 4° C. o/n). The buffer in the filter plate is then removed with suction and the beads are washed with 100 μl of assay buffer II. The latter is then again removed with suction and the beads are resuspended in 100 μl of assay buffer II. This is followed by adding 15-20 ng (up to 200 ng) of the second antigen-specific antibody (biotinylated) in a volume of 10 μl of assay buffer II, and incubation at room temperature on a shaker at 450 revolutions/min over a period of 90 min. This is followed by adding 100 ng of Streptavidin R Phycoerythrin [SAPE] in a volume of 10 μl of assay buffer II. Another incubation is carried out at room temperature on an MTP Shaker at 450 revolutions/min over a period of 30 min, followed by analysis in a reader.

The result shows (FIG. 2) that the signals of the individual analytes were able to be significantly increased. Thus it was possible to generate uniform conditions which enable a plurality of analytes to be measured in a multiplex approach.

1. A method for increasing signal intensity in an assay with simultaneous reduction of unspecific bindings comprising utilizing a reactor buffer comprising a polyvinyl derivative.

2. The method as claimed in claim 1, wherein the polyvinyl derivative comprises polyvinylpyrrolidone and polyvinyl alcohol in the form of a pure polymer, as a copolymer with one another and/or with another suitable comonomer, and/or as a mixture thereof.

3. The method as claimed in claim 2, wherein the copolymer comprises a copolymer of vinyl acetate and/or of vinylpyrrolidone, poly[vinylpyrrolidone-covinyl acetate] and/or poly[vinylpyrrolidone-covinyl alcohol].

4. The method as claimed in claim 1, wherein the molecular weight of the polyvinyl derivative is within a range from 5000 to 500 000 Da.

5. The method as claimed in claim 4, wherein the molecular weight of the polyvinyl derivative is within a range from 24 000 to 360 000 Da.

6. The method as claimed in claim 2, wherein the molecular weight of the polymer or copolymer is within a range from 31 000 to 186 000 Da.

7. The method as claimed in claim 2, wherein the molecular weight of the polyvinyl alcohol alone or in a mixture with polyvinylpyrrolidone is within a range from 9 000 to 500 000 Da.

8. The method as claimed in claim 7, wherein the molecular weight is within a range from 31 000 to 186 000 Da.

9. The method as claimed in claim 8, wherein the molecular weight is within a range from 86 000 to 126 000 Da.

10. The method as claimed in claim 8, characterized in that the molecular weight is within a range from 146 000 to 186 000 Da.

11. The method as claimed in claim 3, wherein the molecular weight of the polyvinylpyrrolidone alone or in combination with polyvinyl alcohol is within a range from 5 000 to 500 000 Da.

12. The method as claimed in claim 11, wherein the molecular weight of the polyvinylpyrrolidone is within a range from 10 000 to 450 000 Da.

13. The method as claimed in claim 12, wherein the molecular weight of the polyvinylpyrrolidone is within a range from 24 000 to 360 000 Da.

14. The method as claimed in claim 1, wherein the concentration of the polyvinyl derivative in said assay is within a range from 0.025 to 5% (w/v).

15. The method as claimed in claim 14, wherein the concentration of the polyvinyl derivative in said assay is within a range from 0.025 to 2% (w/v).

16. The method as claimed in claim 15, wherein the concentration of the polyvinyl derivative in said assay is within a range from 0.1 to 1% (w/v).

17. A method of increasing the signal intensity with simultaneous reduction of unspecific bindings, comprising detecting an analyte bound to a bead and/or to a planar protein array in the presence of a polyvinyl derivative.

18. The method as claimed in claim 17, wherein the capture molecules are applied to a glass, a nitrocellulose, a polyvinylidene fluoride and/or a polystyrene surface.

19. The method as claimed in claim 18, wherein the capture molecule is an antibody, a fragment of an antibody, an aptamer, a peptide and/or an anticalin.

20. A kit for carrying out an immunoassay, comprising at least one buffer comprising a polyvinyl derivative component.

21. The kit as claimed in claim 20, further comprising a capture molecule bound to a bead and/or planar arrays.

22. The kit as claimed in claim 21, wherein the bead and/or planar array comprises a support material of glass, nitrocellulose, polyvinylidene fluoride and/or polystyrene.
